(19) United States
(12) Patent Application Publication
(10) Pub. No.: US 2021/0000912 A1
(43) Pub. Date: Jan. 7, 2021

(54) Compositions And Methods For Prophylaxis Or Treatment Of Pain

(71) Applicant: Interk Peptide Therapeutics Limited, Lane Cove West NSW (AU)

(72) Inventor: Michael Valentine Agrez, Katoomba (AU)

(21) Appl. No.: 16/982,565

(22) PCT Filed: Mar. 20, 2018
(86) PCT No.: PCT/AU2018/050251

§ 371 Date: Sep. 20, 2020

(30) Foreign Application Priority Data
Apr. 2, 2017 (AU) 2017901195

(51) Int. Cl. A61K 38/10; A61P 25/02; A61P 25/04; A61P 29/00; A61K 45/06; A61K 38/08; A61K 47/62

(52) U.S. Cl. CPC A61K 38/10; A61P 25/02; A61P 25/04; A61K 47/62; A61K 45/06; A61K 38/08; A61P 29/00

(57) ABSTRACT

There are described methods for the prophylaxis or treatment of pain in a mammal wherein the method comprising administering to the mammal an effective amount of a pain inhibitor comprising a pain inhibiting peptide having an amino acid sequence of more than 6 contiguous arginine residues, or a physiologically acceptable salt of the peptide. The pain inhibitor can be administered in combination with at least one analgesic and/or at least one anti-inflammatory drug. Pharmaceutical compositions comprising the pain inhibitor together with at least one analgesic and/or at least one anti-inflammatory drug are also provided. The pain may, for example, be selected from one or more of neuropathic pain, inflammatory pain, idiopathic pain and nociceptive pain.

### FIELD OF THE INVENTION

The present invention relates to the prophylaxis or treatment of pain in a mammal and to compounds and compositions for use in the prophylaxis or treatment of pain.

### BACKGROUND TO THE INVENTION

Worldwide, neuropathic and inflammatory pain is a leading cause of poor quality of life.

Neuropathic pain is caused by damage or disease affecting the somatosensory nervous system. Neuropathic pain may be associated with abnormal sensations called dysesthesia or pain from normally non-painful stimuli (allodynia). It may be continuous or episodic and the latter may resemble stabbings or electric shocks. Common qualities include burning or coldness, “pins and needles” sensations, numbness and itching. Neuropathic pain may also result from disorders of the peripheral nervous system or the central nervous system (brain and spinal cord) and in some cases it may be of mixed origin.

At least 8% of the North American and European population is affected by neuropathic pain and in 5% of persons it may be severe (Torrance N et al, J Pain, 2005, 7(4): 281-9; Bouhassira D et al., Pain, 2007, 136(3): 380-7; Fallon M T et al, Support Care Cancer, 2015, 23: 2769-2777). Moreover, only some 40-60% of people may obtain partial relief of neuropathic pain with current treatments.

Central neuropathic pain is found in spinal cord injury, multiple sclerosis and some strokes. Aside from diabetes and other metabolic conditions, the common causes of peripheral painful neuropathies are herpes zoster infection, HIV-related neuropathies, nutritional toxins, other toxins, remote manifestations of malignancies, immune-related disorders, and physical trauma to a nerve trunk. Neuropathic pain is common in cancer as a direct result of cancer on peripheral nerves (e.g., compression by a tumour) or as a side effect of chemotherapy (chemotherapy-induced peripheral neuropathy) (Authier N et al., J Amer. Society of Exp. Neurotherapeutics, 2009, 6: 620-629), radiation injury or surgery (e.g., spinal). Neuropathic pain can also be aggravated by chronic alcoholism and is seen with amputees, spinal disc protrusions, and facial and trigeminal nerve neuralgias. Other causes of chronic pain include spinal diseases such as arachnoiditis, degenerative disc disease, epidural fibrosis, failed back surgery syndrome, lumbar disc herniation, osteoporosis and spinal stenosis. In addition, complex regional pain syndromes are seen after foot or hand surgery, broken bones, or as a result of nerve damage. The result is manifested in two painful syndromes—reflex sympathetic dystrophy and causalgia.

After a peripheral nerve lesion aberrant regeneration may occur with neurons becoming unusually sensitive (peripheral sensitization). In addition, central sensitization is an important mechanism of persistent neuropathic pain. The spinal cord dorsal horn neurons give rise to the spinothalamic tract (STT) which constitutes the major nociceptive pathway. As a consequence of ongoing spontaneous activity arising in the periphery, STT neurons develop increased background activity, enlarged receptive fields and increased responses to afferent stimuli, including normally innocuous tactile stimuli.

It has been reported that more than 230 million surgeries are performed annually worldwide. In the United States, over 50 million inpatient surgeries take place every year, the majority of them in the area of obstetrics and gynaecology, digestive/gastrointestinal tract procedures, and orthopaedics. About half of all patients treated for post-operative pain are not adequately relieved of their pain. Many cases of inadequately managed acute pain progress to chronic pain, and 50-85% of patients undergoing surgery experience moderate to severe pain after the procedure, usually for several days (Gan et al. 2014, Curr Med Res Opin 30:149-160).

Opioid analgesics are commonly used for the treatment of severe pain. In a retrospective study including 37,031 patients that underwent a common surgical procedure, 13.6% of the patients experienced an opioid-related adverse drug event (ORADE). This was associated with a 55% longer length of stay, 47% higher costs of care, 36% increased risk of 30-day readmission, and 3.4 times higher risk of inpatient mortality compared to patients who did not experience an ORADE (Kessler et al. 2013, Pharmacotherapy 33(4); 383-391). In another study with 50 major abdominal surgery patients treated with opioids, 96% of patients reported an ORADE with different levels of severity. In particular, 82% reported at least one side effect that was moderate or severe, while 40% reported at least one side effect that was severe (Gan et al. 2004, Br J Anaesth 92; 681-688).

The most commonly used drugs are opioids, non-steroidal anti-inflammatory drugs (NSAIDs) and local anaesthetics (Bhusal et al. 2016, Drug Deliv Transl Res 6(5): 441-51). Each of these current post-operative pain management (POPM) drugs have a number of dose limiting side-effects. However, opioids still constitute a central role in the management of moderate-to-severe cancer pain (Junej a R, Curr Opin Support Palliative care, 2014, 8(2): 91-101). While there is on-going debate regarding the impact of opioids on increased tumour recurrence (Cata J P et al, Cancer Cell & Microenvironment, 2016, doi: org/10.14800/ccm.1159), it is clear that opioids can cause immunosuppression (Vallejo R et al, Amer. J Therapeutics, 2004, 11(5): 354-365).

Intrathecal morphine is superior to intravenous morphine in patients undergoing minimally invasive cardiac surgery (Mukherjee C et al, Annals of Cardiac Anaesthesia, 2012, 15(2): 122-7). However, intrathecal morphine has been shown to suppress Natural Killer Cell activity after abdominal surgery (Yokota T et al, Canadian J Anaesthesia, 2000, doi: 10/1007/BF03020942). Hence, intrathecal morphine may be problematic in treating patients with chronic pain who are immunosuppressed (Zou W et al, J International Med Res, 2007, 35: 626-36).

Cyclic guanosine monophosphate-(cGMP)-dependent protein kinases (PKGs) exhibit diverse physiological functions in the mammalian system. Two different genes of PKG exist: a) the PKG1 gene that is expressed as cytosolic PKG-1alpha (PKG-1α) and PKG-1 beta (PKG-1β) isoforms, and, b) the PKG2 gene (PRKG2) that expresses the membrane-associated PKG2 protein (Wolerstetter S et al, Pharmceuticals, 2013, 6: 269-286).

PKG-1α mediates the development of many types of chronic pain. PKG-1α is activated in axons at sites of injury or inflammation and subsequently transported in a retrograde fashion to the dorsal root ganglion (DRG) (Luo C et al, PLoS Biol, 2012, 10(3): e1001283). The DRG is part of the peripheral nervous system but communicates directly with the central nervous system. Synaptic long-term potentiation (LTP) enhances the activity of pain centers for extended periods of time, which is the root cause for chronic hyperalgesia (increased sensitivity to pain) and allodynia. This amplified pain signaling has recently been shown to be caused by cGMP-activated kinase 1 (PKG1) (Luo C et al, PLoS Biol, 2012, 10(3): e1001283).

Relevantly, PKG1 is important in causation of inflammatory pain as evidenced by mice lacking PKG1 in which reduced inflammatory hyperalgesia is observed with preservation of acute thermal nociception (Tegeder I et al, PNAS USA, 2004, 101: 3253-3257).

Significantly, activated PKG-1α but not PKG-1β is localised in the neuronal bodies and processes, and is distributed primarily in the superficial laminae of the spinal cord (U.S. Pat. No. 7,294,476). Moreover, PKG-1α expression is significantly increased in the lumbar spinal cord after noxious stimulation (U.S. Pat. No. 7,294,476). Balanol, an anti-fungal metabolite has been shown to modulate neuronal pain via inhibition of PKG (U.S. Pat. No. 8,846,742).

One of the challenges for existing PKG-1α inhibitors is NO/cGMP-mediated cross-talk and activation of Protein Kinase A (PKA) and Protein Kinase C (PKC) (Muller U and Hildebrandt H, J Neuroscience, 2002, 22(19): 8739-8747; Francis S H et al, Pharmacol Review, 2010, 62(3): 525-63; Zeng Z et al, Int J Mol Sci, 2014, 15)6): 10185-98; Bollen E et al, Neuropsychopaharmacology, 2014, 39: 2497-2505).

Recently, Milani et al suggested that poly-arginine peptides may have potential as a neuroprotective therapy for stroke patients based on the observation that polyarginine peptides administered intravenously may reduce brain tissue infarct volume, and hypothesised that the peptides have the capacity to inhibit calcium influx by causing the internalisation of cell surface structures such as ion channels and thereby reduce the toxic neuronal calcium entry that occurs after excitotoxicity and cerebral ischemia (Milani D et al, BMC Neuroscience, 2016, doi: 10.1186/s12868-016-0253-z). Adhesive Tape Removal Tests on the paws of treated rats were performed in that study to assess sensorimotor function but no statistically significant differences were observed between vehicle-treated and peptide treated groups. All poly-arginine peptides tested (R12-R18) were hydroxylated at their C-terminus (Milani et al, vide supra).

It has also been reported that arginine-rich peptides interfere with the function of the NMDA receptors (reviewed in Milani et al, vide supra). NMDA (N-methyl-D-aspartate) receptors (NMDARs) are a subclass of glutamate receptors that require both the binding of glutamate and postsynaptic depolarisation for their activation, and mediate calcium entry when they are activated (Zhou Q & Sheng M, Neuropharmacology, 2013, 74:69-75). NMDRs are known to be present in neurons/synapses of the nociceptive pathway, where NMDRs containing distinct subunit compositions show differential expression patterns (Zhou Q & Sheng M, vide supra). For example, GluN2B-NMDRs, but not GluN2A-NMDRs, are present in C- and A-fibres of the dorsal root ganglia. However, at the spinal cord level, GluN2A-NMDRs are present throughout the dorsal horn except lamina II, while GluN2B-NMDRs appear to be largely absent from lamina II and restricted to certain areas in the superficial dorsal horn (reviewed in Zhou & Sheng, vide supra).

Blocking the function of NMDA receptors has been considered therapeutically impractical as doing so would also impede other vital synaptic transmissions in the central nervous system (Wu H & Tao F, J Biomaterials and Nanobiotechnology, 2011, 2: 596-600). Indeed, the hazards of NMDA receptor blockade on neuronal survival has been well summarised in a review (Hardingham G E and Bading H, TRENDS in Neurosciences, 26: 81-89). Hence, the adverse effects associated with NMDA receptor antagonists have prevented their widespread clinical use (Fisher K et al, J Pain and Symptom Management, 2000, 20(5): 358-373).

### SUMMARY OF THE INVENTION

The present invention stems from the surprising finding that of the Protein Kinase G (PKG) isoforms PKG1α and PKG1β, peptides comprising more than 6 contiguous arginine amino acids as described herein can inhibit at least PKG1α. This finding has application to the prophylaxis or treatment of pain.

In particular, in one aspect of the present invention there is provided a method for prophylaxis or treatment of pain in a mammal, the method comprising administering to the mammal an effective amount of a pain inhibitor comprising a pain inhibiting peptide having an amino acid sequence of more than 6 contiguous arginine residues, or a physiologically acceptable salt of the peptide.

Typically, the peptide comprises at least 9 contiguous arginine residues.

In particularly preferred embodiments, the peptide comprises 12 or more contiguous arginine residues and most preferably, 14 or more contiguous arginine residues.

Typically, the peptide is an inhibitor of the activity of both of PKG1α and PKG1β. In particularly preferred embodiments, the peptide is also an inhibitor of c-Src.

The pain inhibitor can be administered to the mammal alone to reduce or alleviate the pain sensation experienced, or in combination with one or more analgesic and/or anti-inflammatory drugs.

Hence, in another aspect of the invention there is provided a pharmaceutical composition for prophylaxis or treatment of pain, the composition comprising a pain inhibiting peptide having an amino acid sequence of more than 6 contiguous arginine residues, or a physiologically acceptable salt of the peptide, together with a pharmaceutically acceptable carrier.

In at least some embodiments, the composition can further comprise one or more analgesic and/or anti-inflammatory drugs.

In another aspect there is provided a pain inhibiting peptide for use in the prophylaxis or treatment of pain in a mammal, wherein the peptide comprises an amino acid sequence of more than 6 contiguous arginine residues, or a physiologically acceptable salt of the peptide.

In another aspect of the present invention there is provided the use of a pain inhibitor comprising a pain inhibiting peptide in the manufacture of a medicament for the prophylaxis or treatment of pain in a mammal, wherein the peptide comprises an amino acid sequence of more than 6 contiguous arginine residues, or a physiologically acceptable salt of peptide.

Typically, the pain is selected from the group consisting of one or more of neuropathic pain (e.g., central neuropathic pain and/or peripheral neuropathic pain), inflammatory pain (e.g., associated with inflammation responses and/or with release and/or action of pro-inflammatory cytokines such as IL-1β, IL-6 and/or TNF-α), idiopathic pain, nociceptive pain, hyperalgesia, allodynia, pathologic pain, breakthrough pain, incident pain, bone pain, arthritic pain and post-surgery/operative pain. The pain may be chronic pain, acute pain or sub-acute pain.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers, integers or steps.

Any discussion of documents, acts, materials, devices, articles or the like that has been included in this specification is solely for the purpose of providing a context for the invention. It is not to be taken as an admission that any or all of these matters form part of the prior art base or were common general knowledge in the field relevant to the invention as it existed in Australia or elsewhere before the priority date of this application.

The features and advantages of the present invention will become further apparent from the following detailed description of exemplary embodiments of the invention together with the accompanying drawings.

### BRIEF DESCRIPTION OF THE ACCOMPANYING DRAWINGS

FIG. 1 is a graph showing ipsilateral von Frey paw withdrawal threshold (PWT) results for test agents in a chronic constriction injury (CCI) model of neuropathic pain in rats.

FIG. 2 is a histogram showing preferential biodistribution of the peptide IK01400 (RRRRRRRRRRRRRR-NH₂, 14Arg (L-isomer)) and IKD01400 (rrrrrrrrrrrrrr-NH₂, 14Arg (D-isomer)) in murine organs, analysed after 24 hours ex vivo.

FIG. 3 is a graph showing the effect of different dosages of the peptide IK01400 in a mouse Complete Freund's Adjuvant (CFA) model of inflammatory pain compared to morphine.

FIG. 4 is a graph showing grip strength in mice treated with the peptide IK01400 or morphine in the CFA inflammatory pain study.

FIG. 5 is a graph showing pooled results for mice treated with either the IK01400 peptide or morphine from studies performed on three different dates employing the CFA model of inflammatory pain.

### DETAILED DESCRIPTION OF EXEMPLARY EMBODIMENTS OF THE INVENTION

The pain inhibiting peptide in accordance with the invention has an amino acid sequence comprising more than 6 and generally up to 25 contiguous arginine amino acid residues although longer sequences of contiguous arginine residues are not excluded. Thus, the peptide may, for example, comprise a sequence of 7, 8, 9, 10, 11, 12, 13, 14, 15, 16, 17, 18, 19, 20, 21, 22, 23, 24, or 25 contiguous arginine residues. Most usually, the peptide will comprise 20 contiguous arginine residues or less.

Typically, the peptide will have from 8 to 20 contiguous arginine residues, from 8 to 18 contiguous arginine residues and most usually, from 8 to 16 contiguous arginine residues. In particularly preferred embodiments, the peptide will have from 9 to 16 contiguous arginine residues, more usually, will be greater than 12 contiguous arginine residues in length and less than 16 contiguous arginine residues in length. Most preferably, the pain inhibiting peptide is 14 contiguous amino acid residues in length.

Typically, peptides as describe herein are amidated or the like at least their carboxy end to protect against proteolytic degradation. However, any suitable N- or C-terminal modification for protecting against proteolytic degradation can also be employed (e.g., methylation). As described further below, the amino acids of the pain inhibiting peptide can be L-amino acids and/or D-amino amino acids, or other non-naturally occurring amino acids. Thus, the amino acids of an inverted sequence can be all L-amino acids or all D-amino acids, but is not limited thereto.

The use of D-amino acids for the contiguous arginine sequence may be preferable for the treatment of bone pain and osteoarthritis given its preferential uptake in bone when compared with the use of L-amino acids (see FIG. 2).

In at least some embodiments, the pain inhibiting peptide may be coupled to a targeting moiety for targeted delivery of the peptide to target cells (e.g., peripheral neurons) and/or target tissues (e.g., lung, heart or bone tissues). In such embodiments, the targeting moiety can be coupled directly to the N-terminal or C-terminal end of the pain inhibiting peptide or (if provided) via an additional peptide moiety or linker moiety (LM), such as by a peptide or other suitable (e.g., covalent or ionic) bond.

The linker moiety can optionally include or consist of an amino acid sequence coding for one or more enzyme cleavage sites as exemplified below for being enzymatically cleaved at, or in the proximity of, the surface of the target cells or intracellularly within the target cells to release the pain inhibiting peptide.

Typically, the linker moiety comprises a coupling moiety for coupling of the linker moiety to the targeting moiety.

The coupling moiety can comprise any suitable amino acid or amino acid sequence for linkage to the targeting moiety, such as a cysteine (C) amino acid residue (for formation of a disulphide bridge with a terminal cysteine residue provided by the targeting moiety), a lysine residue (K), or a spacer amino acid sequence selected from the group consisting of e.g., KAA, CAA for spacing the lysine (K) or cysteine (C) residue from enzyme cleavage sites (when present) of the linker moiety, wherein A is an alanine amino acid residue. The spacer amino acid sequence can further act as a marker for determination of attachment to the selected targeting moiety. In particularly preferred embodiments, the coupling moiety will comprise one or more β amino acids (e.g., in the case of KAA and CAA the alanine residues can be β amino acids).

The one or more enzyme cleavage sites of a linker moiety (LM) may be selected from the group consisting of cathepsin cleavage sites e.g., GFLGFK (e.g., see Orban et al., Amino Acids, 2011, 41(2):469-483), matrix metalloproteinase (MMP) cleavage sites examples of which include the cleavage sites for MMP-9 and MMP-2 such as GPLGIAGQ, PAGLLGC and GPLGLWAQ (e.g., see Kratz F. et al., Bioorg Med Chem Letters, 2001, 11:2001-2006), and a di-sulfide bridge (—S—S—) cleavable by an intracellular enzyme such as glutathione-s-transferase, the use of all of which is expressly encompassed herein.

Thus, the linker moiety can, for example, comprise or consist of various combinations of coupling moieties and/or enzymatic cleavage sites as described herein, as may be selected from the group consisting of GFLGFK, KAAGFLGFK, CAAGFLGFK, GPLGIAGQ, KAAGPLGIAGQ, CAAGPLGGIAGQ, PAGLLGC, KAAPAGLLGC, CAAPAGLLGC, GPLGLWAQ, KAAGPLGLWAQ and CAAGPLGLWAQ, amongst others. In at least some embodiments, a linker moiety as described herein may comprise more than one enzymatic cleavage site.

Typically, a pain inhibiting peptide in accordance with the invention will have a length in a range of from 7 to about 40 or so amino acids (e.g., 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids).

In still other embodiments, a pain inhibiting peptide for inhibiting PKG1α and/or PKG1β as described herein may be coupled to one or more other pain inhibiting peptides in accordance with the invention by suitable physiologically acceptable scaffolding/framework to form a dimer, multimer or dendrimer. The pain inhibiting peptides in these agents may be the same or comprise two or more different pain inhibiting peptides as described herein.

The entry of a pain inhibitor as described herein into a cell can occur via a number of mechanisms, including via diffusion across an outer cell membrane or by e.g., receptor mediated transport or internalisation, including e.g., via lysosomes which are rich in cathepsin enzyme.

The targeting moiety for the targeting of pain inhibiting peptide as described herein may, for example, be a ligand, a binding peptide, an antibody or binding fragment thereof (such as Fab and F(ab)₂ fragments), or a single-chain variable fragment (scFv), that binds to a receptor or other molecule expressed on the surface of the cells or in the tissue environment of target cells.

Other targeting moieties that may be utilised include morphine glucuronides, enkephalins (e.g., enkephalin pentapeptides such as YGGFL and YGGFM, and d-isomers thereof), NMDA receptor antagonists, transferrin, biotin, folic acid, and hyaluronic acid amongst others, see for instance, Ojima I. et al., Future Med Chem, 2012, 4(1):33-50, the entire contents of which is incorporated herein by cross-reference. Enkephalin which may be utilised further include cyclic, disulphide-bridge containing enkephalins (e.g., bis-penicillamine enkephalins) for binding to delta opioid receptors (Mosberg H I et al, PNAS USA, 1983, 80: 5871-5874)).

Examples of physiological molecules which may be targeted for delivery of pain inhibiting peptide(s) to target cells or tissue in accordance with embodiments of the invention include opioid receptors, NMDA receptors, members of the integrin family and subunits thereof, intercellular adhesion molecules (ICAMs) hormone receptors, neurotransmitter receptors, receptor tyrosine kinase receptors, G-protein linked receptors, growth factor receptors, transmembrane protease receptors, cell-surface proteoglycans, CD44, Fcγ receptors, carcinoembryonic antigen (CEA), hyaluronate receptors, transferrin receptors, folate receptors, glutamate carboxypeptidase II (GCPII), vascular cell adhesion molecules, matrix proteins such as fibronectin, collagen, vitronectin and laminin.

For example, it has been reported that mu opioid receptor endocytosis occurs upon ligand binding (Minnis J G et al., Neuroscience, 2003, 119(1): 33-42). Hence, incorporating a cathepsin cleavage sequence in a pain inhibitor as described herein having a morphine glucuronide targeting moiety may facilitate delivery and intracellular release of the pain inhibiting peptide in neuronal target cells expressing the mu or other opioid receptor.

Moreover, glutamate carboxypeptidase II (GCPII) (also known also as prostate specific membrane antigen (PSMA)) is a membrane antigen present in dorsal root ganglia cells and hydrolyses N-acetylaspartylglutamate to N-acetyl-aspartate and glutamate (Berent-Spillson A et al, J Neurochemistry, 2004, 89: 90-99), and inhibitors of GCPII have been shown to be effective in reducing allodynia (reviewed in Tsukamoto T et al, 2007: http://www.ncbi.nlm.nih.gov/pubmed/17826690.

The cell types targeted in accordance with a method of the invention include neurons (e.g., peripheral sensory neurons), dorsal root ganglion cells and glial satellite cells.

The term “pain inhibitor” as used herein is to be taken in its broadest sense to encompass agents which can exert pain inhibitory activity themselves, and/or wherein pain inhibitory activity is exerted by the pain inhibiting peptide upon release (e.g., by enzymatic cleavage or hydrolysis of bond(s)) from the pain inhibitor.

From the above, it will be understood that in at least some embodiments the pain inhibiting peptide may be conjugated to an antibody or binding fragment thereof to form an antibody-drug conjugate (ADC).

Antibodies or binding fragments thereof (such as Fab, F(ab)₂ and Fv fragments) used as targeting moieties in embodiments of the invention will desirably be specific for the selected target molecule and so will generally comprise monoclonal antibodies or binding fragments thereof. The production of monoclonal antibodies and binding fragments thereof is well known. Chimeric and humanised monoclonal antibodies, and binding fragments of same, are particularly preferred. Chimeric antibodies may, for instance, be provided by substituting the Fc region of a non-human antibody specific for the target molecule with the Fc region of a human antibody. A humanised antibody can be provided by splicing the complementary determining regions (CDRs) in the variable regions of an Fab fragment of a non-human (e.g., mouse, rat, sheep or goat) monoclonal antibody into a human antibody scaffold using recombinant DNA techniques as is also known in the art.

As above, single-chain variable fragment (scFv) and multimeric forms thereof, e.g., bivalent scFvs (e.g., tandem scFvs and diabodies), trivalent scFvs (triabodies) and tetravalent scFvs (tetrabodies), may also be utilised as targeting moieties in embodiments of the invention, with the use of diabodies being particularly preferred. scFvs useful in embodiments in the invention may comprise humanised or native antibody heavy (V_{H}) and light (V_{L}) chains joined together by an amino acid linker sequence (AAL) in either of the two possible orientations V_{L}-AAL-V_{H} or V_{H}-AAL-V_{L}. The length of the linker sequence can vary depending on whether monomeric scFVs, diabodies, triabodies or tetrabodies are to be formed. The linker sequence (AAL) of an scFv will generally be of a length in a range of from about 5 to about 30 amino acids and more generally, in a range of from 5 to 25 amino acids. For formation of diabodies, the linker sequence will generally be about 5 amino acids in length whereby the scFvs are thereby caused to dimerise. For triabodies, the linker sequence may be only 1 or 2 amino acids in length. The design of scFvs, including diabodies, for use in in vivo imaging and therapy is, for example, described in Todorovska A. et al., J Immunol Methods, 2001, Feb. 1; 248(1-2):47-66, Worn A. and Plückthun A., J. Mol. Biol., 2001, 305, 989-1010, and Ahmed Z. A. et al., Clinical and Developmental Immunology, Vol. 2012, Article ID 980250, Hindawi Pub. Corp., the contents of all of which are incorporated herein in entirety by cross-reference.

When a scFv is employed as the targeting moiety, a respective peptide active as described herein can be coupled to a free end of its V_{H} and/or V_{L} chain via a linker moiety comprising one or more enzymatic cleavage sites as described herein. That is, two pain inhibiting peptides as described herein may be coupled to the scFv, one of the peptides being coupled to the free end of the V_{H} chain and the other being coupled to the free end of the V_{L} chain, wherein the pain inhibiting peptides can be the same or different.

Likewise, a pain inhibiting peptide as described herein can be coupled to an antibody, binding fragment(s) thereof, or other targeting moiety via a linker moiety (LM) typically comprising one or more enzyme cleavage sites as described above in any suitable manner.

In yet other embodiments, a pain inhibiting peptide as described herein can be provided incorporated into the targeting moiety itself (e.g., an antibody, antibody binding fragment, or scFv) utilising recombinant DNA techniques. In such embodiments, the pain inhibiting peptide can be coupled to the targeting moiety by a respective coupling moiety comprising enzymatic cleavage site(s) (e.g., a cathepsin cleavage site) at each end of the pain inhibiting peptide, which couple the peptide at each end to the targeting moiety. In such embodiments, the pain inhibiting peptide may be incorporated at any suitable location in the targeting moiety which does not compromise the binding or targeting function of the targeting moiety and which allows for cleavage of the enzymatic cleavage sites in use. For example, a respective pain inhibiting peptide as described herein may be inserted into one or both of the V_{H} and V_{L} chains of an scFv in the manner described above with retention of the binding or targeting function of the scFv, wherein e.g., the pain inhibiting peptide is flanked by enzyme cleavage sequences selected from MMP and other cleavage sequences.

Bi-specific targeting protocols employing more than one targeting moiety for targeting different sites on target tissue or a target cell surface, are also expressly encompassed by the present invention (e.g., bi-specific antibody targeting has recently been review by Weidle U H. et al., Cancer Genomics & Proteomics, 2013, 10: 1-18). That is, a single pain inhibiting peptide as described herein may incorporate two different targeting moieties for targeting different target molecules to one another. As an example, a bi-specific tandem bi-scFv for targeting two different target molecules may be provided e.g., with one or more enzyme e.g., MMP cleavage sequence(s)) between the respective pairs of V_{H} and V_{L} chains.

As another example, a bi-specific antibody which recognises both a target molecule and the glycol moiety of a pegylated pain inhibiting peptide as described herein may be used for targeted delivery of the peptide to the target cell or tissue. The efficacy of such methods to target cancer cells have recently been reported (Howard C B et al., 2016, Adv Healthc Mater, 5(16):2055-68).

Chimeric proteins (i.e., fusion proteins) including a pain inhibiting peptide with or without an attached additional peptide moiety, targeting moiety and/or coupling moiety as described herein, are expressly encompassed as is their use in methods of the invention.

A pain inhibiting peptide or pain inhibitor incorporating a pain inhibiting peptide as described herein can be provided by synthetic or recombinant techniques well known to the skilled addressee and further, can incorporate an amino acid or amino acids not encoded by the genetic code, or amino acid analog(s). For example, one or more D-amino acids, beta-amino acids, and/or homo amino acids may be utilised rather than L-amino acids. Indeed, a peptide or peptide sequence as described herein may consist partly or entirely of D amino acids or combinations of e.g., one or more of D-amino acid(s), beta-amino acid(s), homo amino acid(s), beta-homo amino acid(s), L-amino acid(s), and L- or D-homo amino acids. Examples of beta amino acids include beta-arginine, beta-histidine, beta-lysine, beta-alanine (NH₂—CH₂—CH₂—COOH), beta-phenylalanine, beta-tryptophan, beta-tyrosine, beta-leucine and the like. Examples of homo variant forms of amino acids include homo-cysteine having an additional CH₂ group compared standard L-cysteine. Thus, in some embodiments, the pain inhibiting peptide or pain inhibitor may include L-amino acids, D-amino acids or a mixture of L-amino acids, D-amino acids and/or other amino acid types as described above. The use of peptide(s) including D-amino acids can, for instance, inhibit peptidase activity (e.g., endopeptidases) and thereby enhance stability and increase the half-life of the pain inhibiting peptide or pain inhibitor in vivo.

A pain inhibiting peptide, chimeric protein or other agent comprising a pain inhibiting peptide as described herein may be constrained in a 3-dimensional conformation for use in a method as described herein. For instance, it may be synthesised with side chain structures or otherwise be incorporated into a molecule with a known stable structure in vivo, or be cyclised to provide enhanced rigidity and thereby stability in vivo. Various methods for cyclising peptides, fusion proteins or the like are known. A peptide may be cyclised via four different routes, namely head to tail (C-terminal end to N-terminal end), head to side chain, side chain to tail, or side chain to side chain. For example, a peptide or fusion protein may be provided with two cysteine residues distanced from each other along the peptide or fusion protein and be cyclised by the oxidation of the thiol groups of the residues to form a disulfide bridge between them. Cyclisation may also be achieved by the formation of a peptide bond between N-terminal and C-terminal amino acids of a peptide or for instance, through the formation of a bond between the positively charged amino group on the side chain of a lysine residue and the negatively charged carboxyl group on the side chain of a glutamine acid residue. The formation of direct chemical bonds between amino acids or the use of any suitable linker to achieve cyclisation is also well within the scope of the skilled addressee. A particularly preferred method for achieving cyclisation comprises the formation of a lactam group, and the use of lactamisation to form cyclised forms of peptides and/or peptide actives as described herein is expressly encompassed. Methods for achieving cyclisation including suitable lactamisation methods are, for example, described in White C J and Yudin A K., Contemporary strategies for peptide macrocyclization. Nature Chemistry, June 2011, pp. 509, the entire contents of which is incorporated herein by cross-reference.

A pain inhibiting peptide, chimeric protein or other agent comprising a pain inhibiting peptide as described herein may also include post-translational or post-synthesis modification such as the attachment of carbohydrate moieties or chemical reaction(s) resulting in alkylation or acetylation of amino acid residues or other changes involving the formation of chemical bonds.

The use of peptidomimetics of a pain inhibiting peptide in accordance with the invention is also contemplated and is expressly encompassed herein. A peptidomimetic may, for example, comprise the substitution of one or more of the amino acids of the peptide with an amino acid analogue wherein the amino acid analogue(s) essentially do not diminish the activity of the parent peptide as may be assessed by conventional activity, cell toxicity and/or other suitable assays.

Pain inhibiting peptides, chimeric proteins incorporating a pain inhibiting peptide, or other peptide agent as described herein can be chemically synthesised or produced using conventional recombinant techniques. A nucleic acid encoding a chimeric protein may, for instance, be provided by joining separate cDNA fragments encoding peptides having the desired amino acid sequence(s) by employing blunt-ended termini and oligonucleotide linkers, digestion to provide staggered termini as appropriate, and ligation of cohesive ends. Alternatively, PCR amplification of DNA fragments can be utilised employing primers which give rise to amplicons with complementary termini which can be subsequently ligated together.

Pain inhibiting peptides and chimeric proteins as described herein may be expressed in vitro and purified from cell culture for formulation into suitable pharmaceutical compositions for administration to the mammalian subject.

Solid-phase peptide synthesis (SPPS), click chemistry and Staphylococcal Sortase A mediated peptide-peptide fusion protocols, or combinations of the foregoing, may also be utilised in the provision of a PKG inhibitor as described herein, such as for coupling peptide components together and/or for coupling to a targeting moiety e.g., a scFv etc. Various protocols for such synthesis methods are well known and any suitable such methods may be employed. SPPS methods employing Fmoc or t-Boc or protecting groups for synthesis of a therapeutic agent are particularly preferred. Such synthesis methods are well known and comprise repeated coupling and deprotection cycles with wash steps before and after the deprotection step. In at least some embodiments described herein the entire PKG inhibitor can be synthesised on a solid support by SPSS.

Sortase A (Srt A) is a bacterial enzyme first described in Staphylococcus aureus which cleaves between threonine and glycine in the cleavage sequence LPXTG generating an acyl-enzyme intermediate which can then react with an N-terminal glycine residue to release the enzyme and fuse the glycine and LPXTG tagged components together by a peptide bond, see Levary, D. A et al., “Protein-protein fusion catalysed by sortase A”. PLoS ONE, April 2011, Vol. 6(4):1-6, e18342. See also e.g., Witte, M. D., “Production of unnaturally linked chimeric proteins using a combination of sortase-catalysed transpeptidation and click chemistry”. Nat. Protoc. September 2013, 8(9): 1808-1819, Bently M L. et al., J. Biol Chem, 2008, 283:14762-14771, Mazmanian S K. et al., 1999, Science, 285:760-763, Madej M P et al., Biotechnology and Bioengineering, 2012, 109:1461-1470, and Kornberger P. and Skeria A., 2014, mAbs 6(2): 354-366, the contents of all of the foregoing being incorporated herein in their entirety by cross-reference.

Click chemistry is another high yield method suitable for coupling components together in the provision of pain inhibitors as described herein such as by a metal catalysed (e.g., Cu(I)) azide-alkyne cycloaddition reaction between a terminal azide group on one component and azide group on the other component whereby the components are coupled together by a 1,2,3 triazole bond rather than a peptide bond. 1,2,3 triazole bonds act as a bioisostere to a conventional peptide bond and have the advantage that they are resistant to hydrolysis, see e.g., Li et al., Click chemistry in peptide-based drug design, Molecules, 2013, 18, pp:9797-9817; doi:10.3390/molecu1es18089797. Cyclooctynes such as dibenzo-bicyclo-octyne (DBCO) are likewise highly reactive with azides and offer alternative forms of click chemistry reactions to azide-alkyne cycloadditions as described above, or may be used in combination with azide-alkyne cycloadditions, to provide pain inhibiting peptides and pain inhibitors useful in methods of the invention. Cyclooctyne based click synthesis reactions have the advantage in that they can be carried out without a copper or other metal catalyst.

A targeting moiety such as an scFv, antibody or antibody fragment can, for example, be coupled to a linker moiety (LM) of a pain inhibitor or pain inhibiting peptide as described above by firstly preparing cysteine derivatives of the two components, which are cleaved and purified as HCl salts then coupled to the respective click reagents employing maleimide coupling, exploiting the free sulfhydryl of cysteine in solution phase. The targeting moiety is subsequently derivatized with either the azide or alkyne (or e.g., DBCO) reagent, and click conjugation occurs via the complimentary reagent coupled to the linker moiety.

Pain inhibiting peptides, chimeric proteins, and peptide agents as described herein can be purified from cell culture by sonication or disruption of cell membranes using detergents, centrifugation to remove membrane and solid fragments, and purification from solution or supernatant as applicable by affinity or immunoaffinity chromatography by methods known in the art. Suitable such solid substrates and supports that may be used include, but are not limited to agarose, sepharose and other commercially available supports (e.g., beads of latex, polystyrene, or dextran etc. Antibodies, binding fragments thereof or other suitable binding molecules for immobilizing the peptide or fusion protein of the invention on the solid support for subsequent elution and concentration therefrom can be bound to the solid substrate covalently utilizing commonly employed amide or ester linkers, or by adsorption.

A pain inhibiting peptide or pain inhibitor employed in a method embodied by the invention can have the capacity to translocate across the outer cell membrane of target cells into the cytoplasm and/or nucleus of cells to exert their effect, e.g., by direct penetration across the cell membrane(s), endocytosis-mediated entry, or translocation through the formation of transitory membrane structure(s).

However, methods for targeting a chimeric protein, pain inhibiting peptides or other agents as described herein to target cells and tissues, and/or for facilitating or enhancing cell entry into target cells, include lipid and nanoparticle delivery.

In particular, lipid delivery of pain inhibiting peptides and pain inhibitors in accordance with the invention includes by liposomes, solid lipid nanoparticles, inverse lipid micelles, lipid microtubules and lipid microcylinders (reviewed in Swaminatham J & Ehrhardt C, Expert Opin Drug Deliv, 2012, 9(12): 1489-1503). Liposome containing peptide cargoes have, for example, been proposed for transdermal delivery, for use in nebulisers, for intranasal, ocular and buccal routes and for oral, parenteral and pulmonary routes (reviewed in Swaminatham J & Ehrhardt C, Expert Opin Drug Deliv, 2012, 9(12): 1489-1503). Liposomes have been widely studied as drug, peptide and nucleic acid delivery vehicles (see e.g., International PCT Publication. No. WO 2013/033838), and any such delivery systems may be utilised.

A number of clinically proven liposome-based drug therapies are available, and targeted liposomes compared with non-targeted liposomes achieve enhanced intracellular drug delivery in tumour tissues (Kirpotin D B et al, Cancer Res, 2006, 66: 6732). Moreover, successful liposomal-mediated gene delivery across the blood brain barrier for treatment of gliomas has recently been reported (Yue P-J et al, Molecular Cancer, 2014, 13: 191). Indeed, a combination of liposomal-based cell-targeting and cell-internalisation approaches are available to deliver pain inhibiting peptides, pain inhibitors and other agents as described herein to cells in accordance with methods of the invention.

Approaches for targeting liposomes to cells include the use of liposome-coated cell penetrating peptides CPPs (such as nona-arginine, TAT, and penetratin) (e.g., Malhotra M and Prakash S, Current Nanoscience, 2011, 7: 81-93; Sardan M et al, Faraday Discussions, 2013, 166: 269), liposome conjugated receptor-targeted whole antibodies/antibody fragments, (e.g., scFvs, Fabs (Yu B et al, Mol Membr Biol, 2010, 27(7): 286-298)), and peptide-conjugated liposomes that target receptors such as growth factor receptors or integrins (e.g., utilising RGD peptide or β6 targeting sequence DLXXL), see for instance Nails S et al, Nanomedicine, 2012, 8(6):951-962; Kogelberg H et al, JMB, 2008, 382: 385-401; Chen Z et al, International Journal of Nanomedicine, 2012, 7: 3803-3811; and Gray B P et al, PLOS one, 2013, doi: 10.1371/journal.pone.0072938. Nanoparticles such as liposomes modified with CPPs have increasingly also been recognised as efficient delivery systems for transdermal administration of peptides aided by positive charge binding to skin cells followed by water evaporation that leads to a thin lipid monolayer on the skin surface (Desai P et al, Mol Membr Biol, 2010, 27(7): 247-259).

Besides liposomes, other nanoparticles that may be utilised for delivery of pain inhibiting peptides and pain inhibitors as described herein include nanoparticles such as albumin, gelatine, phospholipids suitable for use in liposomes, polymers, solid metal-containing nanoparticles and the like (e.g., see De Jong W H & Borm P J A, Int J Nanomedicine, 2008, 3(2):133-149). Protocols for externally coating nanoparticles with e.g., antibodies to various ligands or receptors of target cells are also well-recognised. In particular, embodiments for targeting of liposomes accordance with the invention include the use of bispecific antibodies bound to PEG units on the liposome membrane and which also bind to e.g., GCPII membrane antigen on e.g., neuronal target cells.

Fatty acids such as decanoic and dodecanoic acids have also been proposed as means of creating fatty acid peptide salts that exhibit increased transdermal and transmucosal permeability (US 2013/0085105), and all such methods for delivery of pain inhibiting peptides and pain inhibitors as described herein are also expressly encompassed.

In still further embodiments, free terminal end(s) of pain inhibiting peptides and pain inhibitors as described herein may be e.g., methylated, acetylated, or pegylated with a plurality of ethylene glycol monomer units for resistance to degradation by proteases in vivo or to inhibit clearance of the peptide or pain inhibitor from the circulation via the kidneys (e.g., typically by 2 or more monomer units of polyethylene glycol (PEG) and generally, from about 2 to about 11 monomers of PEG (i.e., (PEG)n where n equals from 2 to 11 and is most usually 2). Methods for pegylation of peptides are well known to the skilled addressee.

Whilst a pain inhibiting peptide or pain inhibitor as described herein may be administered to a mammalian subject as the sole drug for the prophylaxis or treatment of pain in accordance with the invention, in at least some embodiments the agent may be administered in combination with one or more other drugs for treatment (i.e., prophylactic or therapeutic) of the pain. Any suitable analgesic and/or anti-inflammatory drugs for the treatment of the pain may be utilised in the combination therapy.

Analgesics that may be used in a pharmaceutically acceptable composition or combination therapy in accordance with the invention may, for instance, be selected from paracetamol, non-steroidal anti-inflammatory drugs (NSAIDs) such as salicylic acids, salicylates, ibuprofen, naproxen, fenoprofen, ketoprofen, and flubiprofen, acetic acid derivatives such as indomethacin, ketorolac and diclofenac, enolic acid derivatives such as piroxicam, meloxicam and tenoxicam, and fenamates such as mefenamic acid, meclofenamic acid, and flufenamicacid acid; COX-2 inhibitors (and particularly, COX-2 selective inhibitors) such as rofecoxib, celecoxib, and etoricoxib; opioids such as morphine, codeine, oxycodone, hydrocodone, dihydromorphone, and pethidine; other analgesics such as flupirtine, gabapentin, pregabalin, enkephalin pentapeptides, conotoxins; analgesic agents that target ion channels, vanilloid receptors, NF-kB/IKB or N-methyl-D-aspartate receptors involved in pain or the propagation of pain; and physiologically acceptable salts and esters of the foregoing. Salicylic acids and salicylates that may be used include those selected from the group consisting of methoxysalicylic acids (e.g., 5-methoxysalicylic acid), aminosalicylic acids (e.g., 4-aminosalicylic acid), and salicylates such as sodium salicylate, benorylate, acetylsalicylic acid (aspirin), methoxy salicylates (e.g., 4-methoxy salicylates, 5-methyl salicylates), methyl salicylates, choline salicylates, choline-magnesium tri salicylates, salicylamides, and bismuth sub salicylates.

Anti-inflammatory drugs that may utilised in a pharmaceutical composition of the invention or be administered in combination with a pain inhibitor in accordance with the invention may, for example, be selected from conventionally used adenosine A2a receptor agonists, glucocorticoids, NSAIDs, and COX2-inhibitors as described above.

A particular example of a pain inhibitor embodied by the invention comprises a targeting moiety (e.g., an enkephalin pentapeptide such as YGGFL) coupled to the N-terminal of a linker moiety including an enzymatic cleavage sequence (e.g., a cathepsin or MMP cleavage sequence) such as a βA-βA-βA-GPLG-IAGQ which is in turn coupled directly to the N-terminal of a polyarginine peptide moiety in accordance with the invention (e.g., a polyarginine peptide comprising 14 contiguous arginine amino acid residues as described herein).

The use of opioids for pain relief is known to cause immunosuppression (Vallejo R et al, Amer. J Therapeutics, 2004, 11(5): 354-365). Immunosuppressed patients or patients for which immunosuppression is undesirable or have a history of adverse reaction(s) to opioids or are otherwise unable to tolerate the administration of opioid(s) (e.g., morphine and morphine derivatives) and whom are in need of pain relief or prophylaxis for pain (e.g., for post-operative pain or chronic pain) may, therefore, particularly benefit from treatment in accordance with a method of the invention and the treatment of such patients is expressly encompassed herein.

The activity and/or cell toxicity profile of a pain inhibiting peptide or pain inhibitor as described herein on target cells and tissues may be determined by various conventionally known assays such as one or more of evaluation of cell morphology, trypan-blue exclusion, assessment of apoptosis, cell proliferation studies (e.g., cell counts, ³H-thymidine uptake and MTT assay), kinase activity assays, Western blot and immunofluorescence studies.

A pain inhibiting peptide, pain inhibitor or other agent in accordance with the invention can be provided in a pharmaceutical composition comprising a pharmaceutically acceptable carrier for administration to the intended subject, and can be administered orally, intranasally, via inhalation (e.g., by aerosol spray), intravenously, parenterally, by intra-articular injection, rectally, subcutaneously, by infusion, topically, intramuscularly, intraperitoneally, intraspinally, intrathecally, epidurally, intraocularly, or via any other route deemed appropriate, and may, for example, be administered pre- and/or post-operatively.

A pharmaceutical composition can, for example, be in the form of a liquid, suspension, emulsion, syrup, cream, ingestible tablet, capsule, pill, suppository, powder, troche, elixir, or other form that is appropriate for the selected route of administration.

Pharmaceutical compositions useful in methods in accordance with the invention include aqueous pharmaceutical solutions. Injectable compositions will be fluid to the extent that syringability exists. Moreover, a pharmaceutically acceptable carrier may comprise or include any suitable conventionally known solvents, dispersion media, physiological saline and isotonic preparations or solutions, surfactants, and any suitable pharmaceutically acceptable carrier (e.g., orally or topically acceptable carriers) may be utilised. Suitable dispersion media can for example contain one or more of ethanol, polyols (e.g., glycerol, propylene glycol, liquid polyethylene glycol and the like), vegetable oils and mixtures thereof.

A pain inhibiting peptide or pain inhibitor as described herein may also be formulated with an inert diluent, an edible carrier and/or it may be enclosed in a hard or soft shell gelatin capsule.

A pharmaceutical composition as described herein can also incorporate one or more preservatives suitable for in vivo and/or topical administration such as parabens, chlorobutanol, phenol, sorbic acid, and thimerosal. In addition, prolonged absorption of the composition may be brought about by the use in the composition of agents for delaying absorption such as aluminium monostearate and gelatin. Tablets, troches, pills, capsules and the like containing an agent as described herein can also contain one or more of the following: a binder such as gum tragacanth, acacia, corn starch or gelatin; a disintegrating agent such as corn starch, potato starch or alginic acid; a lubricant such as magnesium stearate; a sweetening agent such as sucrose, lactose or saccharin; and a flavouring agent.

The use of ingredients and media as described above in pharmaceutical compositions is well known. Except insofar as any conventional media or ingredient is incompatible with a peptide active or pain inhibitor in accordance with the invention, use thereof in therapeutic and prophylactic pharmaceutical compositions as described herein is included.

It is particularly preferred to formulate parenteral compositions in dosage unit form for ease of administration and uniformity of dosage. Dosage unit form as used herein is to be taken to mean a physically discrete unit suited as a unitary dosage for the subject to be treated, each unit containing a predetermined quantity of the pain inhibiting peptide or pain inhibitor in accordance with the invention calculated to produce the desired therapeutic or prophylactic effect in association with the relevant pharmaceutically acceptable carrier used. When the dosage unit form is for example, a capsule, tablet or pill, various ingredients may be used as coatings (e.g., shellac, sugars or both) to the physical form of the dosage unit or to facilitate administration to the mammalian subject.

A pharmaceutical composition used in accordance with a method of the invention will generally contain at least about 1% by weight of a pain inhibiting peptide as described herein. The percentage may be varied and can conveniently be between about 5% to about 80% w/w of the composition or preparation. Again, the amount of the pain inhibiting peptide in accordance with the invention will be such that a suitable effective dosage will be delivered to the subject taking into account the proposed route of administration. Preferred oral pharmaceutical compositions will contain between about 0.1 μg and 15 g of the pain inhibiting peptide.

The dosage of the pain inhibiting peptide in accordance with the invention will depend on a number of factors including whether the pain inhibiting peptide is to be administered for prophylactic or therapeutic use, the severity and nature of the pain, the age of the subject, and related factors including weight and general health of the subject as may be determined by the physician or attendant in accordance with accepted principles. For instance, a low dosage may initially be given which is subsequently increased at each administration following evaluation of the subject's response. Similarly, the frequency of administration may be determined in the same way that is, by continuously monitoring the subject's response between each dosage and if necessary, increasing the frequency of administration or alternatively, reducing the frequency of administration.

Typically, a pain inhibiting peptide or pain inhibitor as described herein will be administered in accordance with a method embodied by the invention to provide a dosage of the pain inhibitor or pain inhibiting peptide in a range of from about 0.0001 mg/kg body weight, 0.001 mg/kg body weight, 0.01 mg/kg body weight, 0.1 mg/kg body weight, or 1.0 mg/kg body weight up to about 300 mg/kg body weight or more. Most preferably, the dosage of the pain inhibitor or pain inhibiting peptide will be 50 mg/kg body weight, 40 mg/kg body weight, 30 mg/kg body weight, 20 mg/kg body weight, 10 mg/kg body weight, or 5 mg/kg body weight or less. When administered orally, up to about 30 g of the pain inhibiting peptide may be administered per day, (e.g., 6 oral doses per day, each dose comprising 5 g of the pain inhibiting peptide).

With respect to intravenous routes, particularly suitable routes are via injection for systemic distribution of the pain inhibiting peptide into blood vessels which supply tissue to be treated. Moreover, the pain inhibiting peptide or the like can be delivered by any suitable infusion or perfusion techniques.

Suitable pharmaceutical acceptable salts of pain inhibitors and pain inhibiting peptides (e.g., amides, acid addition such salts, base addition salts etc.) embodied by the invention that are within a reasonable benefit/risk ratio, pharmacologically effective and appropriate for contact with animal tissues without undue toxicity, irritation or allergic response may be utilised. Representative acid addition salts include hydrochloride, sulfate, bisulfate, maleate, fumarate, succinate, tartrate, tosylate, citrate, lactate, phosphate, oxalate and borate salts. Representative base addition salts include those derived from ammonium, potassium, sodium and quaternary ammonium hydroxides. The salts may include alkali metal and alkali earth cations such a sodium, calcium, magnesium and potassium, as well as ammonium and amine cations. Suitable pharmaceutical salts are well known to the skilled addressee and, for example, are exemplified in S. M Berge et al, J. Pharmaceutical Sciences (1977), the contents of which is incorporated herein in its entirety by cross-reference.

Suitable pharmaceutically acceptable carriers and formulations useful in compositions embodied by the invention as described herein can be found in handbooks and texts well known to the skilled addressee, such as “Remington: The Science and Practice of Pharmacy”, Mack Publishing Co., 1995,

The pain treated in accordance with the invention may be associated with a disease or condition afflicting the mammal. For example, where the pain is bone pain, the pain could be joint or other bone pain arising from bone fracture(s), tumours and bone cancers, osteoporosis, vertebral degeneration, osteomyelitis, septic arthritis, osteoarthritis, and other arthritic conditions.

The mammal treated as described herein may be any mammal treatable in accordance with the invention. For instance, the mammal may be a member of the bovine, porcine, ovine or equine families, a laboratory test animal such as a mouse, rabbit, guinea pig, a cat or dog, or a primate or human. Typically, the mammal is a human.

The invention is further described below by a way of a number of non-limiting Example(s).

### Example 1: Inhibition of PKG1α and PKG1β kinases in a cell free assay

The capacity of test peptides to inhibit the activity of the kinases PKG1α, PKG1β, and Src family kinases in a cell free assay was assessed. The test peptides were amidated at their C-terminal end (indicated by “—NH₂”) unless otherwise noted. Kinase activity assays were conducted by Eurofins Pharma Discovery Services UK Ltd (Dundee Technology Park, Dundee, United Kingdom) and Reaction Biology Corp. (Malvern, Pa., USA).

All test peptides are prepared to 50× final assay concentration in 100% DMSO. This working stock of the test compound is added to assay wells as the first component in the reaction, followed by the remaining components of the respective assay protocols described below. There is no pre-incubation step between the test peptide and the kinase prior to initiation of the reaction. The kinases are diluted in buffer (20 mM MOPS, 1 mM EDTA, 0.01% Brij-35, 5% Glycerol, 0.1% β-mercaptoethanol, 1 mg/mL BSA) prior to addition to the reaction mix.

Positive control wells contain all components of the reaction, except the test peptide of interest. However, DMSO (at a final concentration of 2%) is included in positive control wells to control for solvent effects. Negative control wells contain all components of the reaction, with a reference inhibitor (staurosporine) replacing the test peptide. This abolishes kinase activity and establishes a base-line (0% kinase activity). Kinase assay protocols (Eurofins Pharma Discovery Services UK Ltd, Dundee Technology Park, Dundee, United Kingdom) are described below. Of the Src family kinases, only the assay protocol for c-Src is shown.

### 1. Kinase Assay Protocols

### 1.1 PKG1α (h)

PKG1α (h) is incubated with 8 mM MOPS pH 7.0, 0.2 mM EDTA, 10 μM cGMP, 200 μM peptide RRRLSFAEPG, 10 mM magnesium acetate and [γ-³³P]-ATP (specific activity approx. 500 cpm/pmol, concentration as required). The reaction is initiated by the addition of the MgATP mix. After incubation for 40 minutes at room temperature, the reaction is stopped by the addition of 3% phosphoric acid solution. A 10 μL aliquot of the reaction is then spotted onto a P30 filtermat and washed three times for 5 mins in 75 mM phosphoric acid and once in methanol prior to drying and scintillation counting.

### 1.2 PKG1β (h)

PKG1b(h) is incubated with 8 mM MOPS pH 7.0, 0.2 mM EDTA, 10 μM cGMP, 200 μM RRRLSFAEPG, 10 mM magnesium acetate and [γ-³³P]-ATP (specific activity approx. 500 cpm/pmol, concentration as required). The reaction is initiated by the addition of the MgATP mix. After incubation for 40 minutes at room temperature, the reaction is stopped by the addition of 3% phosphoric acid solution. A 10 μL aliquot of the reaction is then spotted onto a P30 filtermat and washed three times for 5 mins in 75 mM phosphoric acid and once in methanol prior to drying and scintillation counting.

### 1.3 c-Src (h)

c-Src (h) is incubated with 8 mM MOPS pH 7.0, 0.2 mM EDTA, 250 μM KVEKIGEGTYGVVYK (Cdc2 peptide), 10 mM MgAcetate and [γ-³³P]-ATP (specific activity approx. 500 cpm/pmol, concentration as required). The reaction is initiated by the addition of the MgATP mix. After incubation for 40 minutes at room temperature, the reaction is stopped by the addition of 3% phosphoric acid solution. A 10 μL aliquot of the reaction is then spotted onto a P30 filtermat and washed three times for 5 minutes in 75 mM phosphoric acid and once in methanol prior to drying and scintillation counting.

### 2. Results

The results are shown in Tables 1 and 2 below. All results are shown as nanomolar IC₅₀ values. The peptide 11(0140H (RRRRRRRRRRRRRR) is the native peptide consisting of 14 contiguous arginine amino acid residues without any C-terminal amidation or other C-terminal protection.

TABLE 1
Inhibition of PKG1α, PKG1β and c-Src by test peptides

| Peptide | | | PKG1α | PKG1β | c-Src |
|---|---|---|---|---|---|
| code | Short name | Test peptide | IC₅₀ nM | IC₅₀ nM | IC₅₀ nM |
| IK00600 | 6Arg | RRRRRR-NH₂ | >1000 | 457 | 100 |
| IK00800 | 8Arg | RRRRRRRR-NH₂ | 75 | 55 | 26 |
| IK00900 | 9Arg | RRRRRRRRR-NH₂ | 33.5 | 22 | 4 |
| IK00100 | 10Arg | RRRRRRRRRR-NH₂ | 16 | 9 | 7 |
| IK01200 | 12Arg | RRRRRRRRRRRR-NH₂ | 8 | 7 | 6 |
| IK01400 | 14Arg | RRRRRRRRRRRRRR-NH₂ | 6.7 | 5.3 | 5.5 |
| IK0140H | 14Arg OH | RRRRRRRRRRRRRR | 7 | 7 | >1000 |
| IKD01400 | D-isomer | rrrrrrrrrrrrrr-NH₂ | 11 | 10 | 6 |
| | of 14Arg | | | | |
| IK01600 | 16Arg | RRRRRRRRRRRRRRRR-NH₂ | 6 | 4 | 4 |
| IK00200 | 20Arg | RRRRRRRRRRRRRRRRRRRR-NH₂ | 6 | 6 | ND* |

*ND means not determined
(Source: Eurofins Pharma Discovery Services, Dundee, United Kingdom)

In contrast to the 10 mer polyarginine peptide 10Arg, corresponding polylysine, polyhistidine, and polyornithine peptides of the same length failed to show any inhibition of PKG1α or PKG1β activity, as shown below in Table 2.

TABLE 2
Effect of 10 mer poly- lysine, histidine and
ornithine peptides

| Peptide | Short | | PKG1α | PKG1β | c-Src |
|---|---|---|---|---|---|
| code | name | Test peptide | IC₅₀ nM | IC₅₀ nM | IC₅₀ nM |
| IK00100 | 10Arg | RRRRRRRRRR-NH₂ | 16 | 9 | 7 |
| IK00K00 | 10K | KKKKKKKKKK-NH₂ | >1000 | >1000 | 96 |
| IK00010 | 10His | HHHHHHHHHH-NH₂ | >1000 | >1000 | >1000 |
| IKNOR10 | Orn10 | OrnOrnOrnOrnOrnOrn | >1000 | >1000 | 7 |
| | | OrnOrnOrnOrn-NH₂ | | | |

(Source: Eurofins Pharma Discovery Services, Dundee, United Kingdom)

### 3. Discussion

As can be seen from Table 1, inhibition PGK1α and PGK1β was obtained by contiguous arginine sequences tested, with a decrease in IC₅₀ values observed as the length of the test polyarginine sequences increased.

While IC₅₀ values for PKG1α, PKG1β were in the single digit nanomolar range for test peptide RRRRRRRRRRRRRR—NH₂(14 Arg), none of the test peptides comprising deca-lysine (10K), deca-histidine (10H) and deca-ornithine (Orn10) inhibited PKG1α or PKG1β, all of which were also observed to have IC₅₀ values in excess of 1000 nM (Table 2).

### Example 2: Intrathecal Administration of Test Agents in a Neuropathic Pain Model

### 1. Methodology

All animal studies were approved by the Royal North Shore Hospital Animal Ethics Committee (Sydney, Australia) and performed under the guidelines of the Australian code of practice for the care and use of animals for scientific purposes (National Health and Medical Research Council, Australia, 7th Edition). Great care was taken to minimise animal suffering during these studies whenever possible.

### 1.1 Animals

Experiments were performed on male Sprague-Dawley rats weighing 300-400 g at the start of experiments. Animals were housed 2 or 3 per cage until cannula surgery, after which time they were singly housed for 4 days until the day of experiment. Animals were maintained on standard 12-h light/dark cycle with food and water available ad libitum.

### 1.2 Partial Nerve Ligation Surgery

After baseline von Frey measures were taken, animals underwent partial nerve ligation (PNL) surgery, as previously described (Seltzer, Z., Dubner, R., Shir, Y., 1990. A novel behavioural-model of neuropathic pain disorders produced in rats by partial sciatic-nerve injury. Pain 43, 205-218). Briefly, animals were deeply anaesthetised using isoflurane (2.0-2.5% in oxygen), and the sciatic nerve of the left hind-leg was exposed via blunt dissection of the biceps femoris at a site near the trochanter, just distal to the posterior biceps semitendinosus nerve branches of the common sciatic nerve. A 6-0 silk suture was used to tightly ligate the dorsal 30-50% of the nerve. A 4-0 silk suture was used to close the muscle, and VetBond™ tissue adhesive was used to close the skin incision.

### 1.3 Intrathecal catheter surgery

10 days after PNL surgery, rats underwent intrathecal catheter surgery as previously described (Størkson, R. V., Kjørsvik, A., Tjølsen, A., Hole, K., 1996. Lumbar catheterization of the spinal subarachnoid space in the rat. Journal of Neuroscience Methods 65, 167-172). Animals were deeply anaesthetised using isoflurane and incisions were made along the midline at the level of the ventral iliac spines (4-5 cm long) and at the occipital level (0.5 cm long). A 21 G needle was inserted between the L5 and L6 vertebrae and advanced 3-5 mm with the correct localization confirmed by a tail-flick or paw retraction. A 21 cm long catheter (PE tubing, ID 0.2 mm OD 0.5 mm) was advanced through the needle approximately 3 cm rostrally. The catheter was secured to a layer of superficial muscle using 4-0 silk suture, and a 9 cm length of tubing (ID 0.58 mm, OD 0.96 mm) was glued (Loctite 406) to the end of the catheter allowing sufficient diameter for prospective drug administration. When the glue was dry, the catheter was tunneled rostrally using a Bonn micro probe and externalised through the incision made at the occipital region. The skin incision was closed with VetBond™ tissue adhesive and the externalised catheter was secured to the skin with 3-0 silk suture. A small amount of glue was used to secure the suture to the catheter, ensuring no glue was applied to the skin. The dead space of the catheter was filled with 30 μL sterile saline.

### 1.4 Mechanical Allodynia Testing

Mechanical paw withdrawal threshold (PWT) was measured using a series of von Frey hairs with bending pressures ranging from 0.41 to 15.1 g. Rats were placed in elevated plastic cages with wire mesh bases suspended above a table. All rats were given 30 min to acclimatise to the testing environment. Beginning with the 2 g filament, von Frey hairs were pressed perpendicularly against the plantar surface of the left hind paw and held for 2 s. Each von Frey filament was applied seven times at random locations. A positive response was regarded as the sharp withdrawal of the paw, paw licking, or flinching upon removal of the von Frey filament. The mechanical PWT was calculated using the up-down paradigm (Dixon, W. J., 1980. Efficient analysis of experimental-observations. Annual Review of Pharmacology and Toxicology 20, 441-462). If an animal did not respond to any hairs, then the mechanical PWT was assigned as 15 g.

### 1.5 Intrathecal Testing of Analgesic Agents in Nerve Injured Rats

On the day of testing, rats were allowed to acclimatise to the testing chambers for approximately 30 minutes or until settled. Pre-injection (T=0) scores were taken, followed by injection of drug or control (positive and negative). 10 μL of drug or control was washed through the catheter dead-space with 25 μL sterile saline (0.9%) and separated by 2 μL air. The experimenter was blinded to drug treatments throughout testing. After the completion of experiments, catheter patency and placement was confirmed by the injection of lignocaine (8 μL, 2%) and dye (3 μL; 5% Malachite Green) washed through with 15-25 μL sterile saline. Intrathecal lignocaine caused temporary bilateral hind-limb paralysis. After observation of paralysis, animals were immediately sacrificed by CO₂ asphyxiation and spinal cords were exposed to determine the spread of dye. The presence of dye on the dorsal side of lumbar enlargement confirmed catheter placement.

### 2. Results and Discussion

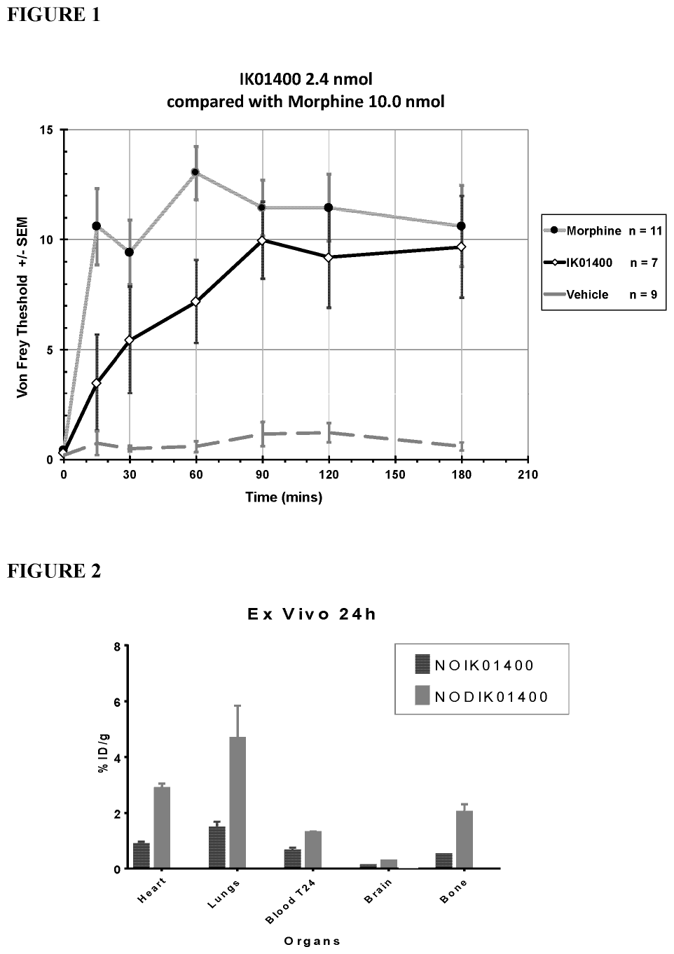

As shown in FIG. 1, the polyarginine peptide H—RRRRRRRRRRRRRR—NH₂ (14 Arg; IK01400) was found to be more effective in alleviating neuropathic pain than morphine on a molar basis when administered intrathecally.

Opioids constitute a central role in the management of moderate-to-severe cancer pain (Juneja R, Curr Opin Support Palliative care, 2014, 8(2): 91-101). While there is on-going debate regarding the impact of opioids on increased tumour recurrence (Cata J P et al, Cancer Cell & Microenvironment, 2016, doi: org/10.14800/ccm.1159) there is no debate about immunosuppression caused by opioids (Vallejo R et al, Amer. J Therapeutics, 2004, 11(5): 354-365). Intrathecal morphine has been shown to be superior to intravenous morphine in patients undergoing minimally invasive cardiac surgery (Mukherjee C et al, Annals of Cardiac Anaesthesia, 2012, 15(2): 122-7). However, intrathecal morphine has also been shown to suppress natural killer cell activity after abdominal surgery (Yokota T et al, Canadian J Anaesthesia, 2000, doi: 10/1007/BF03020942). Hence, intrathecal morphine may be problematic in treating patients with chronic pain who are immunosuppressed (Zou W et al, J International Med Res, 2007, 35: 626-36).

### Example 3: Resistance of L- and D-Isomers of the Peptide 14 Arg to Simulated Gastric Fluid

Test 14 Arg peptides consisting of 14 contiguous L- or D-arginine amino acids were tested for resistance to simulated gastric fluid (porcine pepsin in a HCl and NaCl solution) at a pH of 2.01 for a period of 24 hours. The results are shown in Table 3 and Table 4. Negligible degradation of each peptide was observed by exposure to the simulated gastric fluid over the 24 hour test period.

TABLE 3
Peptide 14 Arg (D-isomer)
H-D-Arg D-Arg D-Arg D-Arg D-Arg D-Arg D-Arg D-Arg D-Arg D-Arg
D-Arg D-Arg D-Arg D-Arg-NH₂

| | | | | |
|---|---|---|---|---|
| Time | 0 | 1 hr | 4 hrs | 24 hrs |
| % of intact peptide observed | 98.79% | 98.92% | 98.70% | 97.53% |

Source: Auspep Pty Limited, Tullamarine, Victoria, Australia

TABLE 4
Peptide 14 Arg (L-isomer)
H-Arg-Arg-Arg-Arg-Arg-Arg-Arg-Arg-Arg-Arg-Arg-Arg-Arg-Arg-NH₂

| | | | | |
|---|---|---|---|---|
| Time | 0 | 1 hr | 4 hrs | 24 hrs |
| % of intact peptide observed | 98.51% | 99.53% | 98.47% | 98.24% |

Source: Auspep Pty Limited, Tullamarine, Victoria, Australia

### Example 4: Positron Emission Tomography—Computed Tomography (PCT-CT) Studies

A bio-distribution study was undertaken using PCT-CT following injection of mice with the peptide 14 Arg comprising either 14 contiguous L-arginine amino acid residues (radio-labelled peptide IK01400, referred to as NOIK01400 in FIG. 2) or 14 contiguous D-amino acid arginine residues (radio-labelled peptide IKD01400, referred to as NODIK01400 in FIG. 2). The study was undertaken at the Centre for Advanced Imaging, Australian Institute for Bioengineering and Nanotechnology, University of Queensland, Australia.

The test peptides were respectively dissolved in MilliQ water at a concentration of 1 mg/mL. No issues with solubility were observed. Peptides were labelled with ⁶⁴Cu at 1000-fold excess of peptide in acetate buffer (˜50 mM, pH 5.5) and gave radio pure products with no free copper detected by radio-TLC. Radio-labelled peptides were diluted with MilliQ water and injected IP in 50 μL of H₂O (containing acetate buffer from the labelling step). Mice were imaged using PET-CT for 45 minutes following injection and then at approximately 8 and 24 hours after injection. Blood samples were taken by tail snip following each imaging time point and activity measured by gamma counter.

After imaging at 24 hours post injection, a final blood sample was collected, mice were euthanized by cervical dislocation and organs (liver, spleen, kidneys, heart, lungs, gut, brain and femur) were collected. Radioactivity of each sample was then measured via gamma counter and the activity present normalized to percent injected dose per gram (% ID/g). The relative presence of the radiolabelled peptides in organs and blood of the mice after 24 hours is shown in FIG. 2.

As can be seen, higher levels of the D-isomer peptide NODIK01400 (columns right side/red) were detected in each of the heart, lungs, blood brain and bone (femur) at the 24 hour time point compared to the L-isomer peptide NOIK01400 (columns left side/blue), with significantly higher levels of the D-isomer peptide NODIK01400 localising in the lungs, heart and bone relative to the level detected in the blood at that time point.

### Example 5: CFA Inflammatory Pain Model and Incapacitance Testing in Mice

A study was undertaken to assess the efficacy of the polyarginine peptide H—RRRRRRRRRRRRRR—NH₂ (14 Arg; IK01400) consisting of a contiguous sequence of 14 L-isomer arginine amino acid residues in a mouse model for inflammatory pain. Mice paws were treated with Complete Freund's Adjuvant (CFA) and following development of inflammation, weight-bearing was assessed for left and right paws commencing 30 minutes after administration of either peptide IK01400, morphine or vehicle control (phosphate buffered saline (PBS), pH 7.4).

### 1. Methods

All experiments involving animals were approved by the University of Sydney Animal Ethics Committee. Experiments were performed under the guidelines of the Australian code of practice for the care and use of animals for scientific purposes (National Health and Medical Research Council, Australia, 7th Edition). Great care was taken to minimise animal suffering during these experiments.

### 1.1 Animals

Experiments were performed on male C57Bl/6 mice weighing 20-25 g. Animals were housed 5 or 6 per cage and maintained on standard 12-h light/dark cycle with food and water available ad libitum. Animals were acclimatised to the testing room, experimenter and testing equipment for at least 5 days prior to testing to reduce novelty- and stress-related confounds.

### 1.2 CFA Inflammatory Pain Model

After baseline incapacitance testing, animals underwent injections of Complete Freund's Adjuvant (CFA) as previously described (Mohammadi, S. and M. J. Christie, α9-nicotinic acetylcholine receptors contribute to the maintenance of chronic mechanical hyperalgesia, but not thermal or mechanical allodynia. Molecular Pain, 2014. 10: p. 9). Animals were anaesthetised using isoflurane (2.0-2.5% in oxygen) and the left hindpaw was swabbed with ethanol. 50 μL CFA was injected subcutaneously into the plantar surface of the left hindpaw using a 30 G needle. Animals were allowed to recover in the home-cage and tested 3 days later. No paw drooping or autotomy was observed in any CFA-injected animals.

### 1.3. Test Compound Molar Equivalents

The test compounds were administered via intra-peritoneal injection. The amounts of each test compound administered and the molar equivalents for test peptide IK01400 and morphine are shown in Table 5.

TABLE 5
Molar equivalents for test compounds

| Test | Amount | Amount | Molar equivalent |
|---|---|---|---|
| compound | (μg) | (mg/kg) | (μM) |
| IK01400 | 200 | 8.61 | 454 |
| IK01400 | 100 | 4.31 | 227 |
| Morphine | 240 | | 3,729 |

### 1.4 Incapacitance Testing

Differences in weight bearing across the injured and uninjured hindpaws were assessed using the Linton Incapacitance tester (MJS Technology Ltd., Hertfordshire, UK) (Clayton, N. M., et al., Validation of the dual channel weight averager as an instrument of the measurement of clinically relevant pain. British Journal of Pharmacology, 1997. 120: p 219). Mice were placed into a transparent acrylic chamber that has an upward sloping wall fore-wall that encourages the mice to assume an upright posture. The floor of the chamber comprised of two electronic weighing scales and measurements are taken when each hind-paw rested symmetrically on each scale. This enables quantification of postural changes of the animal reflecting spontaneous pain by independent measurement of the weight that the animal applies to each hind paw. Equal weight is applied to each hind paw by the mouse in the absence of any perception of pain difference between the paws whilst a lower weight is applied to the injured one of the paws. The average of six measurements were taken, and represented as a ratio of ipsilateral (left hind-paw) to contralateral (right hind-paw) weight distribution. Thus, decreased ratios indicated less weight placed on the ipsilateral side and greater pain score.

### 1.4 Grip Strength Testing

Forepaw grip strength was determined using a grip strength meter (DFIS-2 series digital force gauge, Columbus Instruments, Columbus, Ohio). The animals' forelimbs were placed on a horizontal bar connected to a force gauge. The animal was the gently pulled away from the bar and the peak force required for a release was recorded. Each mouse was tested 3 times, with a 1 min inter-trial interval (Martinez-Huenchullan, S. F., et al., “Utility and reliability of non-invasive muscle function tests in high-fat-fed mice”. Experimental Physiology, 2017. 102(7): p. 773-778). The average peak tension (g), and tension normalised to body weight were compared.

### 2. Results

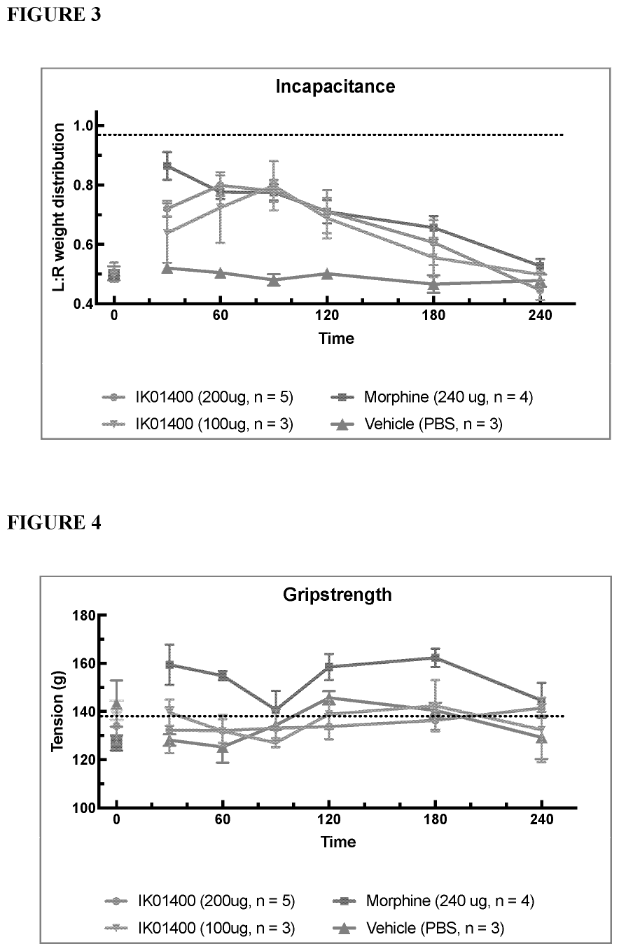

As shown in FIG. 3, incapacitance (pain felt) is similar to morphine at the two doses of IK01400 (100 μg and 200 μg). Relevantly though, the molar equivalence of the doses of the peptide utilised is markedly less than that of morphine, with the molar equivalence of 100 μg of IK01400 being approximately one-sixteenth that of morphine. The dotted line represents the average baseline scores for pain-free animals before CFA injection. Error bars are standard errors.

To confirm that pain relief was not due to paralysis of paws, grip-strength was determined for all compounds including vehicle (PBS) (see FIG. 4). The dotted line represents the average baseline scores for pain-free animals before CFA injection. Error bars are standard errors. There was no difference between vehicle control treated animals and animals receiving IK01400, and animals were alert at all time points. The increased grip strength seen for morphine-treated animals was due to the side effects of morphine i.e., hyperactivity.

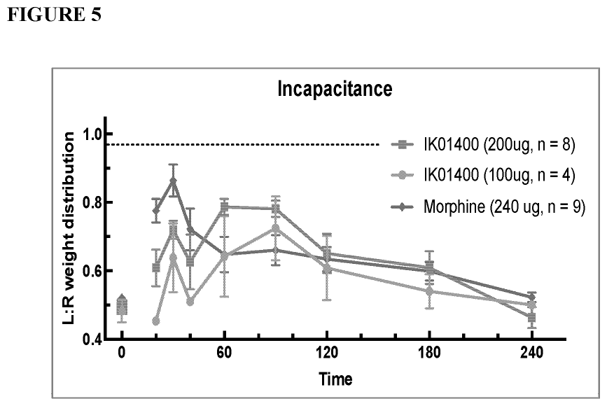

To determine whether there was a dose response effect for IK01400, data were pooled for experiments performed on three different dates (23 Jun. 2017, 21 Jul. 2107 and 15 Sep. 2017) as shown in FIG. 5. As can be seen, morphine appears more effective during the first 30 minutes after injection but less effective than IK01400 (8.6 mg/kg) during the second hour of testing.

### 3. Discussion

The results show that the IK01400 (14Arg) peptide alleviated pain in the mouse model of inflammatory pain utilised at both doses of the peptide employed, with the peptide having particular effect during the second hour after administration. At a dose of 8.6 mg/kg the peptide was shown to be more effective than morphine during the second hour after administration. That the peak effect of the peptide was reached after 1 hour following administration whilst the effect of morphine was reduced during this period is indicative the peptide may be used alone or in a combination treatment with opioids for pain relief. Moreover, unlike morphine which was observed to induce hyperactivity in the mice, no hyperactivity was observed in the mice treated with the peptide.

Although a number of embodiments of the invention have been described above it will be understood that various modifications and changes may be made thereto without departing from the invention. The above described embodiments are therefore only illustrative and are not to be taken as being restrictive.

1. A method for prophylaxis or treatment of pain in a mammal, the method comprising administering to the mammal an effective amount of a pain inhibitor comprising a pain inhibiting peptide having an amino acid sequence of more than 6 contiguous arginine residues, or a physiologically acceptable salt of the peptide.

2. The method according to claim 1, wherein the peptide comprises at least 9 contiguous arginine residues.

3. The method according to claim 1, wherein the peptide comprises at least 12 contiguous arginine residues.

4. The method according to claim 1, wherein the peptide comprises 14 or more contiguous arginine residues.

5. The method according to claim 4, wherein the peptide comprises 14 contiguous arginine amino acids.

6. The method according to any one of claims 1 to 5, wherein the peptide is C-terminal protected.

7. The method according to claim 6, wherein the peptide is amidated at its C-terminal end.

8. The method according to any one of claims 1 to 7, wherein the peptide is an inhibitor of the activity of PKG1α.

9. The method according to claim 8, wherein the peptide is an inhibitor of PKG1α and PKG1β.

10. The method according to any one of claims 1 to 9, wherein the peptide is an inhibitor of c-Src.

11. The method according to any one of claims 1 to 10, wherein one or more of the arginine amino acid residues are D-isomer amino acids.

12. The method according to claim 11, wherein all of the arginine amino acid residues are D-isomer amino acids.

13. The method according to any one of claims 1 to 6, wherein the peptide consists of the contiguous arginine amino acids.

14. The method according to any one of claims 1 to 13, wherein the peptide is coupled to a targeting moiety for targeted delivery of the peptide to target cells.

15. The method according to claim 14, wherein the peptide is coupled to the targeting moiety via a linker moiety.

16. The method according to claim 15, wherein the linker moiety comprises amino acid sequence coding for one more enzyme cleavage sites for being cleaved for release of the peptide.

17. The method according to any one of claims 1 to 16, wherein the pain inhibiting peptide is administered in combination with at least one analgesic and/or at least one anti-inflammatory drug.

18. The method according to any one of claims 1 to 17, wherein the pain is selected from the group consisting of one or more of neuropathic pain, inflammatory pain, idiopathic pain, nociceptive pain, hyperalgesia, allodynia, pathologic pain, breakthrough pain, incident pain, bone pain, arthritic pain and post-surgery/operative pain.

19. The method according claim 18, wherein the pain comprises neuropathic pain and/or inflammatory pain.

20. A pharmaceutical composition comprising a pain inhibitor together with at least one analgesic and/or at least one anti-inflammatory drug, wherein the pain inhibitor is as defined in any one of claims 1 to 19.

21. The composition according to claim 20, wherein the analgesic is selected from the group consisting of paracetamol, non-steroidal anti-inflammatory drugs (NSAIDs), COX-2 selective inhibitors, opioids, flupirtine, pregabalin, enkephalin pentapeptides, conotoxins, and analgesic agents that target ion channels, vanilloid receptors, NF-kB/IKB or N-methyl-D-aspartate receptors involved in pain or the propagation of pain.

22. The composition according to claim 20, wherein the anti-inflammatory drug is selected from the group consisting of adenosine A2a receptor agonists, glucocorticoids, NSAIDs, and COX2-inhibitors.

23. A pain inhibiting peptide for use in the prophylaxis or treatment of pain in a mammal, wherein the peptide comprises an amino acid sequence of more than 6 contiguous arginine residues, or a physiologically acceptable salt of the peptide.

24. The use of a pain inhibiting peptide in the manufacture of a medicament for the prophylaxis or treatment of pain in a mammal, wherein the peptide comprises an amino acid sequence of more than 6 contiguous arginine residues, or a physiologically acceptable salt of peptide.
